(19) United States
(12) Patent Application Publication
(10) Pub. No.: US 2022/0241472 A1
(43) Pub. Date: Aug. 4, 2022

(54) RED BLOOD CELL FILTERING APPARATUS AND SYSTEM COMPRISING THE SAME

(71) Applicant: Taiwan RedEye, Biomedical Inc., Hsinchu City (TW)

(72) Inventors: Shuo-Ting YAN, Hsinchu City (TW); Ya-Ling Chiang, Hsinchu City (TW)

(21) Appl. No.: 17/166,625

(22) Filed: Feb. 3, 2021

(51) Int. Cl. A61M 1/02; B01D 69/02; B01D 69/06

(52) U.S. Cl. CPC A61M 1/0281; B01D 69/02; B01D 69/06; B01D 2325/04; A61M 2202/0415; B01D 2325/02; A61M 2202/0429

(57) ABSTRACT

Provided is a red blood cell filtering apparatus comprising: a filtering film having multiple through holes; and a shell having a first opening on an upper portion of one lateral part of the shell, and a second opening on a lower portion of the other lateral part of the shell; the filtering film is accommodated in the shell. Also provided is a red blood cell filtering system comprising the red blood cell filtering apparatus, a first blood collector and a second blood collector; wherein the first blood collector has a first connecting opening, and the first connecting opening is connected with the first opening through the first connecting apparatus; the second blood collector has a second connecting opening, and the second connecting opening is connected with the second opening through the second connecting apparatus.

### BACKGROUND OF THE INVENTION

### 1. Field of the Invention

The present invention relates to a red blood cell filtering apparatus, especially a red blood cell filtering apparatus which can rapidly filter red blood cells to obtain platelet-rich plasma. The present invention also relates to a red blood cell filtering system comprising the red blood cell filtering apparatus.

### 2. Description of the Prior Arts

The main components of human blood comprise red blood cells, white blood cells, platelets and plasma. Red blood cells (also known as erythrocytes) do not have nuclei, and they have a diameter of about 6 micrometers (μm) to 8 μm. For adults, the red blood cell count for men is about 5,000,000 per microliter (μL) of blood to 6,000,000 per μL of blood and that for women is about 4,000,000 per μL of blood to 5,000,000 per μL of blood. White blood cells (also known as leukocytes) have nuclei, and they have a diameter of about 8 μm to 15 μm. White blood cells are part of the immune system, and they can pass through the space between capillary vessel cells by amoeboid movement and get into tissues. For adults, the white blood cell count is about 4,000 per μL of blood to 11,000 per μL of blood. Platelets have a diameter of about 2 μm to 3 μm. For adults, the platelet count is about 150,000 per μL of blood to 400,000 per μL of blood. Platelets contain abundant active growth factors which are helpful in angiogenesis, tissue regeneration and repair.

Platelet-rich plasma (PRP) is a blood-based product rich in growth factors, and it can be applied to a variety of parts in the human body, and has broad applications in regenerative medicine. In addition, platelet-rich plasma can be prepared by a patient's own blood, so the risk of rejection is extremely low. Thus, autologous platelet-rich plasma has a great medical potential.

Conventionally, the preparation of autologous platelet-rich plasma includes: collecting blood; optionally mixing the blood with an anticoagulant; and removing red blood cells by centrifugation. To prepare platelet-rich plasma sufficient for one basic therapy at present, the blood sampling process needs to be run twice and the centrifugation process needs to be run once or twice, and the preparation takes about 15 to 30 minutes. The hospital needs to purchase expensive centrifugation equipment to provide the therapy for patients, and this will increase the cost of this medical service, and lower a patient's intention to take autologous platelet-rich plasma therapy. Therefore, if the cost and time for the preparation of autologous platelet-rich plasma can be reduced, a better medical therapy can be provided for patients in need of long-term care and regenerative medicine, thereby bringing good business opportunities.

### SUMMARY OF THE INVENTION

To overcome the shortcomings, the present invention provides a red blood cell filtering apparatus and a red blood cell filtering system comprising the apparatus, which can rapidly and directly filter out red blood cells in blood to obtain platelet-rich plasm ready for injection, without using any centrifuge equipment nor involving any blood handling step (including transferring blood samples from one area to another area). Therefore, the cost and time for preparation of autologous platelet-rich plasma can be effectively reduced, and the contamination risk of blood samples caused by any blood handling step (such as transferring) can also be reduced.

Thus, one objective of the present invention is to provide a red blood cell filtering apparatus comprising: a filtering film having multiple through holes, in which any of the through holes has a diameter of 3.00 μm to 5.00 μm on the front side of the filtering film; and a shell having a first opening on an upper portion of one lateral part of the shell, and a second opening on a lower portion of the other lateral part of the shell, and the lateral part and the other lateral part are opposite to each other; wherein the filtering film is accommodated in the shell, and the front side of the filtering film faces to an inner side of a top wall of the shell. Since the size of any of the through holes on the front side of the filtering film is smaller than the diameter of red blood cells, red blood cells can be directly filtered out to obtain platelet-rich plasma.

Another objective of the present invention is to provide a red blood cell filtering system comprising the above-mentioned red blood cell filtering apparatus, a first blood collector and a second blood collector; wherein the first blood collector has a first connecting opening, and a first needle part or a first connecting apparatus is mounted to the first connecting opening, and when the first connecting apparatus is mounted to the first connecting opening, the first connecting opening is connected with the first opening through the first connecting apparatus; the second blood collector has a second connecting opening, and a second needle part or a second connecting apparatus is mounted to the second connecting opening, and when the second connecting apparatus is mounted to the second connecting opening, the second connecting opening is connected with the second opening through the second connecting apparatus; and when the second connecting opening is connected with the second opening through the second connecting apparatus, the interior of the second blood collector is in a vacuum state. With the red blood cell filtering system, a patient's blood is collected by the first blood collector, and then the red blood cell filtering apparatus connects the first blood collector and the second blood collector by the first connecting apparatus and the second connecting apparatus, respectively. During connection, the vacuum state inside the second blood collector results in a pressure difference between the front side and reverse side of the filtering film, so the blood in the first blood collector will be filtered by the filtering film, and the resulting platelet-rich plasma will be collected in the second blood collector.

The advantages of the present invention include that the autologous platelet-rich plasma can be rapidly prepared by the red blood cell filtering apparatus and the red blood cell filtering system, and applied to the patient himself/herself directly. No expensive centrifugation device is needed. In addition, there is no additional contamination risk of blood samples caused by any blood handling step (such as transferring).

In some embodiments of the present invention, the front side of the filtering film refers to the side contacting blood first and filtering red blood cells comprised therein; and the reverse side of the filtering film is the other side which is opposite to the front side.

In some embodiments of the present invention, the multiple through holes are arranged at equal intervals. In the present invention, the phrase “arranged at equal intervals” means that the spacing distances between the centers of any two adjacent through holes are the same. In some embodiments of the present invention, the spacing distance between the centers of any two adjacent through holes is 65 μm to 75 μm, 68 μm to 72 μm, or 70 μm to 71 μm.

In some embodiments of the present invention, the through holes have a diameter of 3.00 μm to 5.00 μm, 3.20 μm to 4.80 μm, 3.40 μm to 4.60 μm, 3.60 μm to 4.40 μm, 3.80 μm to 4.20 μm, 3.90 μm to 4.10 μm, 3.95 μm to 4.05 μm, or 4.00 μm to 4.02 μm on the front side of the filtering film.

In some embodiments of the present invention, the through holes have a diameter of 30.00 μm to 40.00 μm, 32.00 μm to 38.00 μm, 34.00 μm to 36.00 μm, 34.50 μm to 35.50 μm, or 35.00 μm on the reverse side of the filtering film.

In some embodiments of the present invention, the filtering film is made of a biocompatible metal or a biocompatible plastic. In some embodiments of the present invention, the biocompatible metal may be titanium, a nickel-titanium alloy or a cobalt-nickel alloy, but not limited thereto. In some embodiments of the present invention, the biocompatible plastic may be polydimethylsiloxane (PDMS), poly(methyl methacrylate) (PMMA) or polyethylene terephthalate (PET), but not limited thereto.

In some embodiments of the present invention, the first opening is located on the top wall of the shell. In some embodiments of the present invention, the first opening is located on the side wall of the shell.

In some embodiments of the present invention, the second opening is located on the bottom wall of the shell.

In some embodiments of the present invention, the filtering film is accommodated in the shell, to separate the shell into the upper portion and the lower portion. In some embodiments of the present invention, the upper portion of the shell has a height of 2 millimeters (mm) to 5 mm.

In some embodiments of the present invention, the first connecting apparatus is fixed between the red blood cell filtering apparatus and the first blood collector, so the red blood cell filtering apparatus is fixed on the first blood collector.

In the present invention, the vacuum state inside the second blood collector is an imperfect vacuum state or a perfect vacuum state.

In some embodiments of the present invention, the upper portion of the shell further extends outward from the other lateral part of the shell, to form an additional accommodating space. In some embodiments of the present invention, the accommodating space can be used to accommodate part of the filtered-out red blood cells.

In some embodiments of the present invention, the red blood cell filtering apparatus further has an accommodating part, and the shell further has a third opening on the upper portion of the other lateral part of the shell, and the accommodating part is mounted to the third opening. In some embodiments of the present invention, the third opening is located on the top wall of the shell. In some embodiments of the present invention, the third opening is located on the side wall of the shell. In some embodiments of the present invention, the red blood cell filtering apparatus further has a third connecting apparatus, and the accommodating part is connected with the third opening through the third connecting apparatus. In some embodiments of the present invention, when the accommodating part is connected with the third opening through the third connecting apparatus, the interior of the accommodating part is not in a vacuum state. In some embodiments of the present invention, when the accommodating part is connected with the third opening through the third connecting apparatus, the interior of the accommodating part is in a vacuum state. In the present invention, the vacuum state inside the accommodating part is an imperfect vacuum state or a perfect vacuum state. In some embodiments of the present invention, when the interior of the accommodating part is in a vacuum state, the volume of the accommodating part is smaller than that of second blood collector, so the suction resulted from the accommodating part is weaker than that resulted from the second blood collector.

In some embodiments of the present invention, the first connecting apparatus and the second connecting apparatus each independently comprise a puncturing part and a separating part. In the present invention, the phrase “each independently comprise” means “separately and optionally comprise” herein. In some embodiments of the present invention, the first connecting apparatus comprises a first puncturing part and a first separating part. In some embodiments of the present invention, the second connecting apparatus comprises a second puncturing part and a second separating part.

In some embodiments of the present invention, the puncturing part comprises a needle part. In the present invention, the term “needle part” refers to a hollow-bore needle through which blood can pass, and the size of the needle part is chosen in accordance with the red blood cell filtering apparatus, the first blood collector or the second blood collector.

In some embodiments of the present invention, the separating part is a sealing part. In some embodiments of the present invention, the separating part comprises a penetrable separating film. In some embodiments of the present invention, the first separating part and the second separating part each independently comprise a penetrable separating film. In some embodiments of the present invention, the penetrable separating film can be penetrated by a puncturing part, and the film restores to its original state after taking out of the puncturing part. In some embodiments of the present invention, the penetrable separating film is a rubber film. In some embodiments of the present invention, the rubber film is made of a butyl rubber, such as chlorinated butyl rubber, or brominated butyl rubber.

In some embodiments of the present invention, the first blood collector further has a blood inlet, and the first needle part or a first stopper is mounted to the blood inlet. In some embodiments of the present invention, when the first blood collector simultaneously has a first connecting opening and a blood inlet, the first needle part is mounted to the blood inlet. In some embodiments of the present invention, when the first needle part is mounted to the first connecting opening, the first connecting opening is not connected with the first connecting apparatus. In one embodiment of the present invention, the first needle part is mounted to the first connecting opening or the blood inlet first for blood sampling; then the first connecting opening is connected with the first opening through the first connecting apparatus. In some embodiments of the present invention, a first transferring tube is further mounted between the first connecting opening and the first needle part; and/or, one first transferring tube is further mounted between the blood inlet and the first needle part. In some embodiments of the present invention, the first blood collector is a syringe or a blood bag.

In some embodiments of the present invention, the second blood collector further has a blood outlet, and the second needle part or a second stopper is mounted to the blood outlet. In some embodiments of the present invention, when the second blood collector simultaneously has a second connecting opening and a blood outlet, the second needle part is mounted to the blood outlet. In some embodiments of the present invention, when the second needle part is mounted to the second connecting opening, the second connecting opening is not connected with the second connecting apparatus. In one embodiment of the present invention, the second connecting opening is connected with the second opening through the second connecting apparatus first for filtration, and the platelet-rich plasma obtained after filtration is collected into the second blood collector; then the second needle part is mounted to the second connecting opening or the blood outlet, to inject or extract the collected platelet-rich plasma. In another embodiment of the present invention, the second connecting opening is connected with the second opening through the second connecting apparatus for filtration; then an injector is used to extract the platelet-rich plasma collected in the second blood collector through the injector's needle part, and the platelet-rich plasma is moved into the injector for later injection. In one embodiment of the present invention, the second connecting apparatus comprises a second puncturing part and a second separating part, and the injector's needle part penetrates the second separating part to extract the platelet-rich plasma collected in the second blood collector. In one embodiment of the present invention, the second connecting apparatus is removed first, and then the injector's needle part passes through the second connecting opening to extract the platelet-rich plasma collected in the second blood collector. In some embodiments of the present invention, a second transferring tube is further mounted between the second connecting opening and the second needle part; and/or, one second transferring tube is further mounted between the blood outlet and the second needle part. In some embodiments of the present invention, the second blood collector is a syringe, a blood bag or a test tube.

In some embodiments of the present invention, the size of the filtering film is decided according to the volume of the first blood collector, especially to the maximum blood volume of the first blood collector. In some embodiments of the present invention, the first blood collector has a volume of 20 milliliters (mL) to 25 mL; namely, the maximum blood volume of the first blood collector is 20 mL to 25 mL. In some embodiments of the present invention, when the first blood collector has a volume of 20 mL to 25 mL, the filtering film has a width of 12 mm to 15 mm, a length of 12 mm to 15 mm, and a thickness of 10 μm to 50 μm. In some embodiments of the present invention, the first blood collector and the second blood collector are each independently a 25 mL syringe with a maximum blood volume of 20 mL to 25 mL.

### BRIEF DESCRIPTION OF THE DRAWINGS

FIG. 1 is the schematic diagram showing the arrangement of the through holes on the filtering film of the red blood cell filtering apparatus of the present invention.

FIG. 2 is the sectional view of one through hole on the filtering film of the red blood cell filtering apparatus of the present invention.

FIG. 3 is the schematic diagram showing the structure of the red blood cell filtering apparatus of the present invention.

FIGS. 4A to 4C are schematic diagrams of one embodiment of the red blood cell filtering system of the present invention; wherein FIG. 4A shows the connection between the first blood collector and the first needle part; FIG. 4B shows the connection of the red blood cell filtering system of the present invention during filtration; FIG. 4C shows the connection between the second blood collector and the second needle part.

FIGS. 5A to 5C are schematic diagrams of one embodiment of the red blood cell filtering system of the present invention; wherein FIG. 5A shows the connection between the first blood collector and the first needle part; FIG. 5B shows the connection of the red blood cell filtering system of the present invention during filtration; FIG. 5C shows the connection between the second blood collector and the second needle part.

FIGS. 6A to 6C are the schematic diagrams of one embodiment of the red blood cell filtering system of the present invention; wherein FIG. 6A shows the connection between the first blood collector and the first needle part; FIG. 6B shows the connection of the red blood cell filtering system of the present invention during filtration; FIG. 6C shows the connection between the second blood collector and the second needle part.

FIG. 7 is the schematic diagram of one embodiment of the red blood cell filtering system of the present invention.

### DETAILED DESCRIPTION OF THE PREFERRED EMBODIMENTS

To further illustrate the technical means of the present invention for achieving the objectives, the embodiments of the invention are described in detail below with reference to the accompanying drawings. It should be noted that the invention is not limited to the specific embodiments described herein.

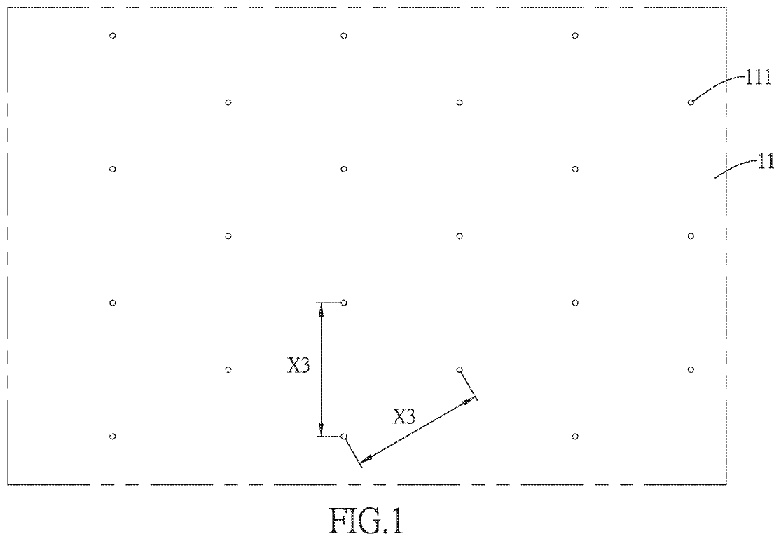

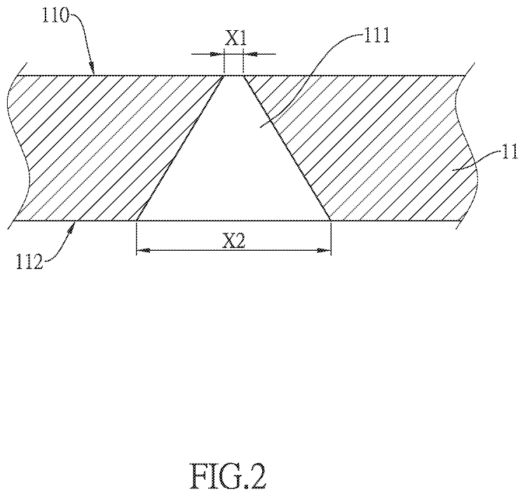

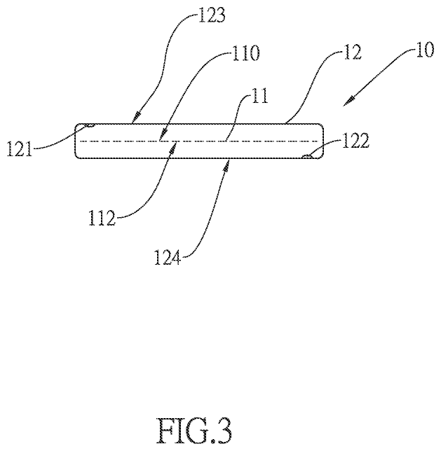

As shown in FIGS. 1 to 3, the red blood cell filtering apparatus 10 of the present invention comprises a filtering film 11 and a shell 12. As shown in FIG. 1, the filtering film 11, which is made of cobalt-nickel alloy, has multiple through holes 111 arranged at equal intervals. Also, as shown in FIGS. 1 and 2, the through holes 111 have a diameter X1 of 3.0 μm to 5.0 μm on the front side 110 of the filtering film 11, a diameter X2 of 30.0 μm to 40.0 μm on the reverse side 112 of the filtering film 11, and a spacing distance X3 between the centers of two adjacent through holes 111 of 71 μm.

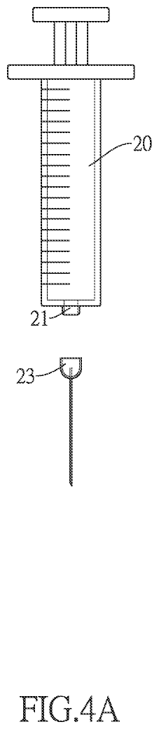

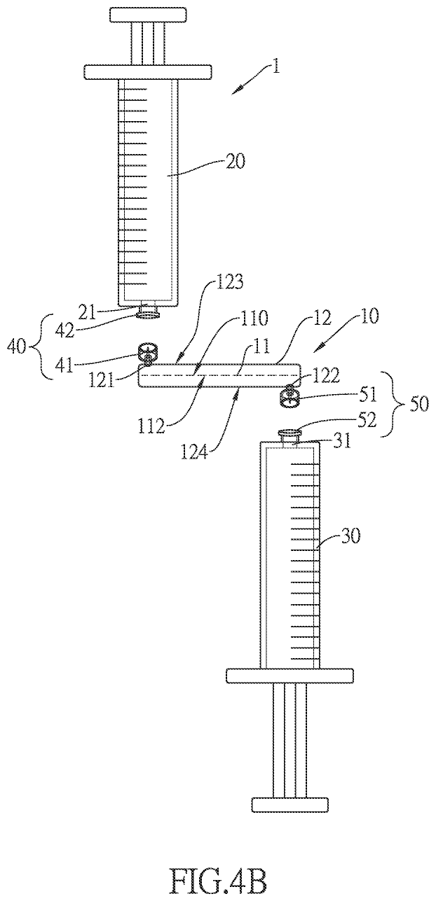

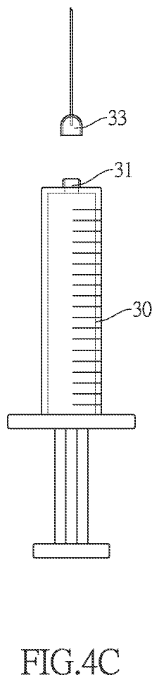

FIGS. 4A to 4C show one embodiment of the red blood cell filtering system 1 of the present invention.

As shown in FIG. 4B, the red blood cell filtering system 1 comprises a red blood cell filtering apparatus 10, a first blood collector 20, a second blood collector 30, a first connecting apparatus 40 and a second connecting apparatus 50, wherein the first blood collector 20 and the second blood collector 30 are each a 25 mL syringe. The shell 12 of the red blood cell filtering apparatus 10 has a first opening 121 and a second opening 122 which are opposite to each other, wherein the first opening 121 is located on the top wall 123 of one lateral part of the shell 12, and the second opening 122 is located on the bottom wall 124 of the other lateral part of the shell 12. The filtering film 11 is accommodated in the shell 12, and the front side 110 of the filtering film 11 faces to the inner side of the top wall 123.

As shown in FIGS. 4A and 4B, the first blood collector 20 has a first connecting opening 21, and the first connecting opening 21 and the first opening 121 are connected by the first connecting apparatus 40. The first connecting apparatus 40 comprises a first puncturing part 41 and a first separating part 42. For the first connecting opening 21, the first separating part 42 (as shown in FIG. 4B) or a first needle part 23 (as shown in FIG. 4A) can be mounted thereon. First, as shown in FIG. 4A, the first needle part 23 is mounted to the first connecting opening 21 for blood sampling from a patient. Then the first needle part 23 is removed, and the first separating part 42 is mounted to the first connecting opening 21, as shown in FIG. 4B. After that, the first puncturing part 41 mounted to the first opening 121 penetrates the first separating part 42, such that the first connecting opening 21 and the first opening 121 are connected by the first connecting apparatus 40, and the first blood collector 20 and the red blood cell filtering apparatus 10 are connected.

As shown in FIGS. 4B and 4C, the second blood collector 30 has a second connecting opening 31, and the second connecting opening 31 and the second opening 122 are connected by the second connecting apparatus 50. The second connecting apparatus 50 comprises a second puncturing part 51 and a second separating part 52. For the second connecting opening 31, the second separating part 52 (as shown in FIG. 4B) or a second needle part 33 (as shown in FIG. 4C) can be mounted thereon. First, as shown in FIG. 4B, the second separating part 52 is mounted to the second connecting opening 31, and the second blood collector 30 is brought into a vacuum state. When the first blood collector 20 and the red blood cell filtering apparatus 10 are being connected, or after they are connected, the second puncturing part 51 penetrates the second separating part 52, such that the second connecting opening 31 and the second opening 122 are connected by the second connecting apparatus 50, and the second blood collector 30 in a vacuum state and the red blood cell filtering apparatus 10 are connected. After that, the second blood collector 30 will make a pressure difference in the red blood cell filtering system 1, and this pressure difference will make the blood in the first blood collector 20 flow into the red blood cell filtering apparatus 10 through the first connecting apparatus 40 for filtration.

Since the front side 110 of the filtering film 11 faces to the inner side of the top wall 123, and the diameter of the through holes 111 on the front side 110 of the filtering film 11 is smaller than the diameter of red blood cells, red blood cells in the blood will be filtered out and stay on the filtering film 11. Platelets rich in growth factors have a diameter smaller than the diameter of the through holes 111, so they will pass through the through holes 111 with plasma, and get into the second blood collector 30 through the second connecting apparatus 50. In addition, white blood cells are capable of amoeboid movement, so they may pass through the through holes 111, and get into the second blood collector 30 through the second connecting apparatus 50.

After filtration, the platelet-rich plasma in which red blood cells are removed is collected in the second blood collector 30. The second separating part 52 is removed from the second connecting opening 31, and the second needle part 33 is mounted to the second connecting opening 31, as shown in FIG. 4C, such that the fresh platelet-rich plasma can be injected into the patient.

Additionally, the second blood collector 30 can be a test tube in a vacuum state for blood collection, in which the collected platelet-rich plasma can be stored therein for a period of time. When it is for use, a new injector is used to extract the platelet-rich plasma and inject the platelet-rich plasma into the patient.

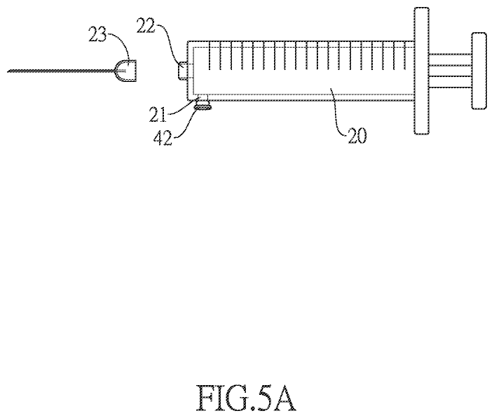

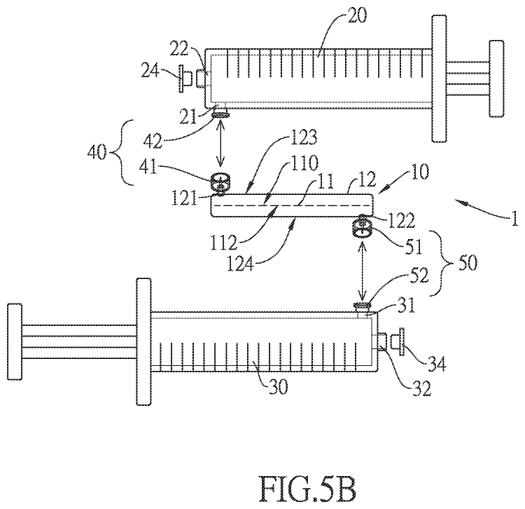

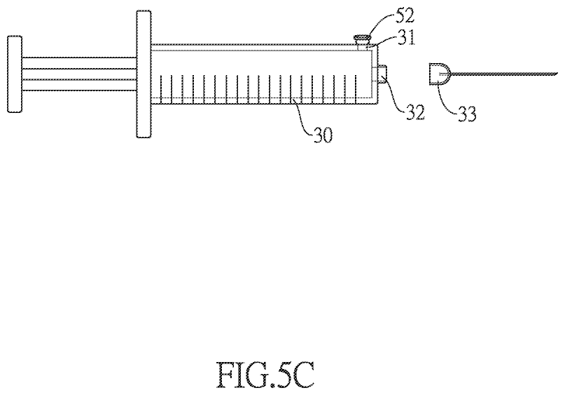

FIGS. 5A to 5C show another embodiment of the red blood cell filtering system 1 of the present invention.

As shown in FIG. 5B, the red blood cell filtering system 1 comprises a red blood cell filtering apparatus 10, a first blood collector 20, a second blood collector 30, a first connecting apparatus 40 and a second connecting apparatus 50, wherein the first blood collector 20 and the second blood collector 30 are each a 25 mL syringe. The shell 12 of the red blood cell filtering apparatus 10 has a first opening 121 and a second opening 122 which are opposite to each other, wherein the first opening 121 is located on the top wall 123 of one lateral part of the shell 12, and the second opening 122 is located on the bottom wall 124 of the other lateral part of the shell 12. The filtering film 11 is accommodated in the shell 12, and the front side 110 of the filtering film 11 faces to the inner side of the top wall 123.

As shown in FIGS. 5A and 5B, the first blood collector 20 simultaneously has a first connecting opening 21 and a blood inlet 22, and the first connecting opening 21 and the first opening 121 are connected by the first connecting apparatus 40. The first connecting apparatus 40 comprises a first puncturing part 41 and a first separating part 42. For the blood inlet 22, a first stopper 24 (as shown in FIG. 5B) or a first needle part 23 (as shown in FIG. 5A) can be mounted thereon. First, as shown in FIG. 5A, the first needle part 23 is mounted to the blood inlet 22 for blood sampling from a patient. Then the first needle part 23 is removed, and the first stopper 24 is mounted to the blood inlet 22, as shown in FIG. 5B. After that, the first puncturing part 41 mounted to the first opening 121 penetrates the first separating part 42, such that the first connecting opening 21 and the first opening 121 are connected by the first connecting apparatus 40, and the first blood collector 20 and the red blood cell filtering apparatus 10 are connected.

As shown in FIGS. 5B and 5C, the second blood collector 30 simultaneously has a second connecting opening 31 and a blood outlet 32, and the second connecting opening 31 and the second opening 122 are connected by the second connecting apparatus 50. The second connecting apparatus 50 comprises a second puncturing part 51 and a second separating part 52. For the blood outlet 32, a second stopper 34 (as shown in FIG. 5B) or a second needle part 33 (as shown in FIG. 5C) can be mounted thereon. First, as shown in FIG. 5B, the second stopper 34 is mounted to the blood outlet 32, and the second blood collector 30 is brought into a vacuum state. When the first blood collector 20 and the red blood cell filtering apparatus 10 are being connected, or after they are connected, the second puncturing part 51 penetrates the second separating part 52, such that the second connecting opening 31 and the second opening 122 are connected by the second connecting apparatus 50, and the second blood collector 30 in a vacuum state and the red blood cell filtering apparatus 10 are connected. After that, the second blood collector 30 will make a pressure difference in the red blood cell filtering system 1, and this pressure difference will make the blood in the first blood collector 20 pass through the first connecting apparatus 40 and flow into the red blood cell filtering apparatus 10 for filtration.

Since the front side 110 of the filtering film 11 faces to the inner side of the top wall 123, and the diameter of the through holes 111 on the front side of the filtering film 11 is smaller than the diameter of red blood cells, red blood cells in the blood will be filtered out and stay on the filtering film 11. Platelets rich in growth factors have a diameter smaller than the diameter of the through holes 111, so they will pass through the through holes 111 with plasma, and get into the second blood collector 30 through the second connecting apparatus 50. In addition, white blood cells are capable of amoeboid movement, so they may pass through the through holes 111, and get into the second blood collector 30 through the second connecting apparatus 50.

After filtration, the platelet-rich plasma in which red blood cells are removed is collected in the second blood collector 30. The second stopper 34 is removed from the blood outlet 32, and the second needle part 33 is mounted to the blood outlet 32, as shown in FIG. 5C, such that the fresh platelet-rich plasma can be injected to the patient.

Additionally, the second blood collector 30 can be a test tube in a vacuum state for blood collection, in which the collected platelet-rich plasma can be stored for a period of time. When it is for use, a new injector is used to extract the platelet-rich plasma and inject the platelet-rich plasma into the patient.

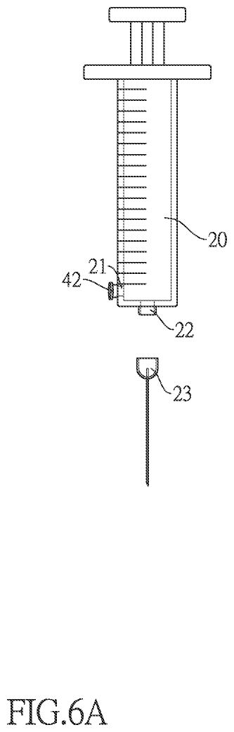

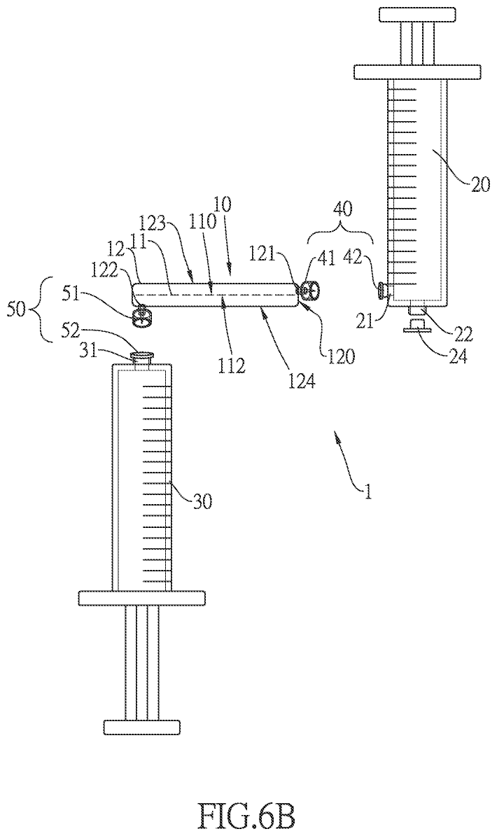

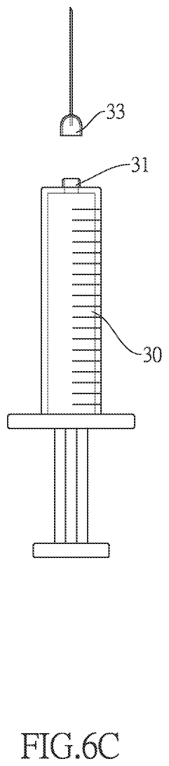

FIGS. 6A to 6C show another embodiment of the red blood cell filtering system 1 of the present invention.

As shown in FIG. 6B, the red blood cell filtering system 1 comprises a red blood cell filtering apparatus 10, a first blood collector 20, a second blood collector 30, a first connecting apparatus 40 and a second connecting apparatus 50, wherein the first blood collector 20 and the second blood collector 30 are each a 25 mL syringe. The red blood cell filtering apparatus 10 has a filtering film 11 and a shell 12, the filtering film 11 is accommodated in the shell 12, and the front side 110 of the filtering film 11 faces to the inner side of the top wall 123. The shell 12 of the red blood cell filtering apparatus 10 has a first opening 121 and a second opening 122, wherein the first opening 121 is located on the side wall 120 of one lateral part of the shell 12 at a position higher than the filtering film 11, and the second opening 122 is located on the bottom wall 124 of the other lateral part of the shell 12.

As shown in FIGS. 6A and 6B, the first blood collector 20 simultaneously has a first connecting opening 21 and a blood inlet 22, and the first connecting opening 21 and the first opening 121 are connected by the first connecting apparatus 40. The first connecting apparatus 40 comprises a first puncturing part 41 and a first separating part 42. For the blood inlet 22, a first stopper 24 (as shown in FIG. 6B) or a first needle part 23 (as shown in FIG. 6A) can be mounted thereon. First, as shown in FIG. 6A, the first needle part 23 is mounted to the blood inlet 22 for blood sampling from a patient. Then the first needle part 23 is removed, and the first stopper 24 is mounted to the blood inlet 22, as shown in FIG. 6B. After that, the first puncturing part 41 mounted to the first opening 121 penetrates the first separating part 42, such that the first connecting opening 21 and the first opening 121 are connected by the first connecting apparatus 40, and the first blood collector 20 and the red blood cell filtering apparatus 10 are connected.

As shown in FIGS. 6B and 6C, the second blood collector 30 has a second connecting opening 31, and the second connecting opening 31 and the second opening 122 are connected by the second connecting apparatus 50. The second connecting apparatus 50 comprises a second puncturing part 51 and a second separating part 52. For the second connecting opening 31, the second separating part 52 (as shown in FIG. 6B) or a second needle part 33 (as shown in FIG. 6C) can be mounted thereon. First, as shown in FIG. 6B, the second separating part 52 is mounted to the second connecting opening 31, and the second blood collector 30 is brought into a vacuum state. When the first blood collector 20 and the red blood cell filtering apparatus 10 are being connected, or after they are connected, the second puncturing part 51 penetrates the second separating part 52, such that the second connecting opening 31 and the second opening 122 are connected by the second connecting apparatus 50, and the second blood collector 30 in a vacuum state and the red blood cell filtering apparatus 10 are connected. After that, the second blood collector 30 will make a pressure difference in the red blood cell filtering system 1, and this pressure difference will make the blood in the first blood collector 20 pass through the first connecting apparatus 40 and flow into the red blood cell filtering apparatus 10 for filtration.

Since the front side 110 of the filtering film 11 faces to the inner side of the top wall 123, and the diameter of the through holes 111 on the front side 110 of the filtering film 11 is smaller than the diameter of red blood cells, red blood cells in the blood will be filtered out and stay on the filtering film 11. Platelets rich in growth factors have a diameter smaller than the diameter of the through holes 111, so they will pass through the through holes 111 with plasma, and get into the second blood collector 30 through the second connecting apparatus 50. In addition, white blood cells are capable of amoeboid movement, so they may pass through the through holes 111, and get into the second blood collector 30 through the second connecting apparatus 50.

After filtration, the platelet-rich plasma in which red blood cells are removed is collected in the second blood collector 30. The second separating part 52 is removed from the second connecting opening 31, and the second needle part 33 is mounted to the second connecting opening 31, as shown in FIG. 6C, such that the fresh platelet-rich plasma can be injected on the patient.

Additionally, the second blood collector 30 can be a test tube in a vacuum state for blood collection, in which the collected platelet-rich plasma can be stored for a period of time. When it is for use, a new injector is used to extract the platelet-rich plasma and inject the platelet-rich plasma into the patient.

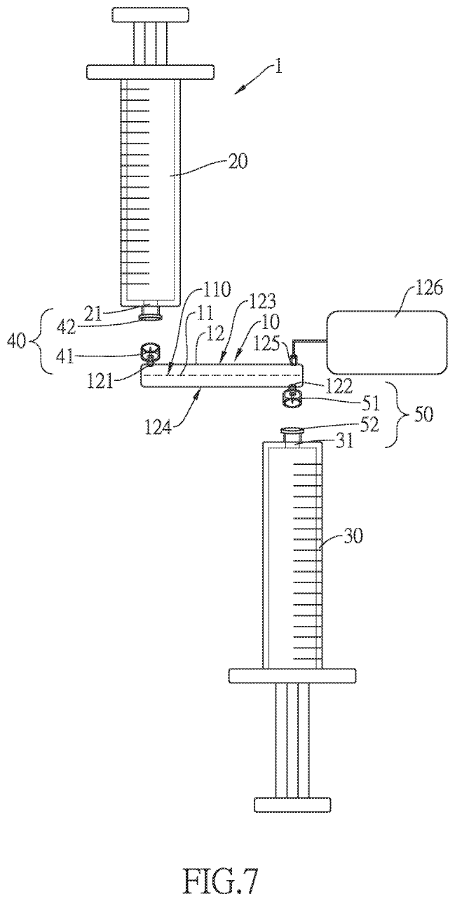

FIG. 7 shows another embodiment of the red blood cell filtering system 1 of the present invention.

As shown in FIG. 7, the red blood cell filtering system 1 comprises a red blood cell filtering apparatus 10, a first blood collector 20, a second blood collector 30, a first connecting apparatus 40 and a second connecting apparatus 50, wherein the first blood collector 20 and the second blood collector 30 are each a 25 mL syringe. The shell 12 of the red blood cell filtering apparatus 10 has a first opening 121 and a second opening 122 which are opposite to each other, wherein the first opening 121 is located on the top wall 123 of one lateral part of the shell 12, and the second opening 122 is located on the bottom wall 124 on the other lateral part of the shell 12. In addition, the red blood cell filtering apparatus 10 further has a third opening 125 located on the top wall 123, wherein the third opening 125 is opposite to the second opening 122 located on the bottom wall 124, and an accommodating part 126 is mounted to the third opening 125 to accommodate red blood cells after filtration. The third opening 125 and the accommodating part 126 also can be applied in other embodiments of the present invention.

In the embodiments shown in FIGS. 4A to 4C, FIGS. 5A to 5C, FIGS. 6A to 6C, and FIG. 7, when the first blood collector 20 is a 25 mL syringe, the blood sampling and filtration can be completed in 4 minutes. Therefore, in the present invention, a patient's autologous platelet-rich plasma can be prepared without any anticoagulant by using the red blood cell filtering apparatus 10 of the present invention, and can be applied to the patient immediately. However, an appropriate amount of an anticoagulant also can be pre-loaded in the first blood collector 20 to ensure that the blood will not be clotted during the preparation of platelet-rich plasma.

When the blood samples of normal adults were filtered by the red blood cell filtering system 1 of the present invention to obtain platelet-rich plasma, the red blood cells comprised in the platelet-rich plasma were counted by routine processes, and it was found that the red blood cell count was lower than 2,000 per μL of blood, which was much lower than that in normal blood (i.e., 4,000,000 per μL of blood to 6,000,000 per μL of blood), indicating that red blood cells were effectively filtered out. In addition, the platelets comprised in the platelet-rich plasma were also counted by routine processes, and it was found that the platelet count was higher than 1,000,000 per μL of blood, which was much higher than that in normal blood (i.e., 150,000 per μL of blood to 400,000 per μL of blood). Therefore, the red blood cell filtering apparatus of the present invention and the system thereof can effectively and directly filter out red blood cells, and can be used for the preparation of autologous platelet-rich plasma.

What is claimed is:

1. A red blood cell filtering apparatus, comprising:
a filtering film, having multiple through holes, any of the through holes having a diameter of 3.00 μm to 5.00 μm on a front side of the filtering film; and
a shell, having a first opening on an upper portion of one lateral part of the shell, and a second opening on a lower portion of the other lateral part of the shell, the lateral part and the other lateral part being opposite to each other; wherein
the filtering film is accommodated in the shell, and the front side of the filtering film faces to an inner side of a top wall of the shell.

2. The red blood cell filtering apparatus as claimed in claim 1, wherein the filtering film is made of a biocompatible metal or a biocompatible plastic.

3. The red blood cell filtering apparatus as claimed in claim 1, wherein the first opening is located on the top wall or a side wall of the shell.

4. The red blood cell filtering apparatus as claimed in claim 1, wherein the second opening is located on a bottom wall of the shell.

5. The red blood cell filtering apparatus as claimed in claim 1, wherein the red blood cell filtering apparatus further has an accommodating part, and the shell further has a third opening on an upper portion of the other lateral side, and the accommodating part is mounted to the third opening.

6. The red blood cell filtering apparatus as claimed in claim 5, wherein the red blood cell filtering apparatus further has a third connecting apparatus, and the accommodating part is connected with the third opening through the third connecting apparatus.

7. A red blood cell filtering system, comprising the red blood cell filtering apparatus as claimed in claim 1, a first blood collector and a second blood collector; wherein the first blood collector has a first connecting opening, a first needle part or a first connecting apparatus is mounted to the first connecting opening, and when the first connecting apparatus is mounted to the first connecting opening, the first connecting opening is connected with the first opening through the first connecting apparatus; wherein the second blood collector has a second connecting opening, a second needle part or a second connecting apparatus is mounted to the second connecting opening, and when the second connecting apparatus is mounted to the second connecting opening, the second connecting opening is connected with the second opening through the second connecting apparatus; and when the second connecting opening is connected with the second opening through the second connecting apparatus, an interior of the second blood collector is in a vacuum state.

8. The red blood cell filtering system as claimed in claim 7, wherein the first connecting apparatus and the second connecting apparatus each independently comprise a puncturing part and a separating part.

9. The red blood cell filtering system as claimed in claim 7, wherein the first blood collector further has a blood inlet, and the first needle part or a first stopper is mounted to the blood inlet.

10. The red blood cell filtering system as claimed in claim 7, wherein the second blood collector further has a blood outlet, and the second needle part or a second stopper is mounted to the blood outlet.

11. The red blood cell filtering system as claimed in claim 7, wherein the first blood collector is a syringe or a blood bag; the second blood collector is a syringe, a blood bag or a test tube.

12. The red blood cell filtering system as claimed in claim 7, wherein when the first blood collector has a volume of 20 mL to 25 mL, the filtering film has a width of 12 mm to 15 mm, a length of 12 mm to 15 mm, and a thickness of 10 μm to 50 μm.

13. A red blood cell filtering system, comprising the red blood cell filtering apparatus as claimed in claim 5, a first blood collector and a second blood collector; wherein the first blood collector has a first connecting opening, a first needle part or a first connecting apparatus is mounted to the first connecting opening, and when the first connecting apparatus is mounted to the first connecting opening, the first connecting opening is connected with the first opening through the first connecting apparatus; wherein the second blood collector has a second connecting opening, a second needle part or a second connecting apparatus is mounted to the second connecting opening, and when the second connecting apparatus is mounted to the second connecting opening, the second connecting opening is connected with the second opening through the second connecting apparatus; and when the second connecting opening is connected with the second opening through the second connecting apparatus, an interior of the second blood collector is in a vacuum state.

14. The red blood cell filtering system as claimed in claim 13, wherein the first connecting apparatus and the second connecting apparatus each independently comprise a puncturing part and a separating part.

15. The red blood cell filtering system as claimed in claim 13, wherein the first blood collector further has a blood inlet, and the first needle part or a first stopper is mounted to the blood inlet.

16. The red blood cell filtering system as claimed in claim 13, wherein the second blood collector further has a blood outlet, and the second needle part or a second stopper is mounted to the blood outlet.

17. The red blood cell filtering system as claimed in claim 13, wherein the first blood collector is a syringe or a blood bag; the second blood collector is a syringe, a blood bag or a test tube.

18. The red blood cell filtering system as claimed in claim 13, wherein when the first blood collector has a volume of 20 mL to 25 mL, the filtering film has a width of 12 mm to 15 mm, a length of 12 mm to 15 mm, and a thickness of 10 μm to 50 μm.
